(19) United States
(12) Patent Application Publication
(10) Pub. No.: US 2003/0199523 A1
(43) Pub. Date: Oct. 23, 2003

(54) Heterocyclic calcium in channel blockers

(76) Inventor: Terrance P. Snutch, Vancouver (CA)

Correspondence Address: Kate H. Murashige, Morrison & Foerster LLP, Suite 500, 3811 Valley Centre Drive, San Diego, CA, 92130, US

(21) Appl. No.: 10/377,090

(22) Filed: Feb. 28, 2003

Related U.S. Application Data
(60) Provisional application No. 60/360,917, filed on Feb. 28, 2002.

(51) Int. Cl. A61K 31/506; A61K 31/444

(52) U.S. Cl. 514/256; 514/332; 514/336

(57) ABSTRACT

Compounds comprising at least one aromatic ring linked to a heterocycle are described which are useful in altering abnormal calcium channel activity.

### CROSS-REFERENCE TO RELATED APPLICATION

This application claims benefit under 35 U.S.C. § 119(e) to provisional application No. 60/360,917 filed Feb. 28, 2002. The contents of this application are incorporated herein by reference.

### TECHNICAL FIELD

The invention relates to compounds useful in treating conditions associated with abnormal calcium channel function. More specifically, the invention concerns compounds containing substituted or unsubstituted derivatives of 5-membered heterocyclic moieties that are useful in treatment of conditions such as stroke and pain.

### BACKGROUND ART

PCT publication WO 01/45709 published Jun. 28, 2001 discloses calcium channel blockers where a piperidine or piperazine ring links a benzhydril moiety to an additional aromatic moiety or benzhydril. This publication is incorporated herein by reference. As explained in this publication, native calcium channels have been classified by their electrophysiological and pharmacological properties as T, L, N, P and Q types. T-type (or low voltage-activated) channels describe a broad class of molecules that transiently activate at negative potentials and are highly sensitive to changes in resting potential. The L, N, P and Q-type channels activate at more positive potentials (high voltage activated) and display diverse kinetics and voltage-dependent properties. There is some overlap in biophysical properties of the high voltage-activated channels, consequently pharmacological profiles are useful to further distinguish them. Whether the Q- and P-type channels are distinct molecular entities is controversial. Several types of calcium conductances do not fall neatly into any of the above categories and there is variability of properties even within a category suggesting that additional calcium channels subtypes remain to be classified.

Biochemical analyses show that neuronal high voltage activated calcium channels are heterooligomeric complexes consisting of three distinct subunits (α₁, α₂δ, and β). The α₁ subunit is the major pore-forming subunit and contains the voltage sensor and binding sites for calcium channel antagonists. The mainly extracellular α₂ is disulfide-linked to the transmembrane δ subunit and both are derived from the same gene and are proteolytically cleaved in vivo. The β subunit is a nonglycosylated, hydrophilic protein with a high affinity of binding to a cytoplasmic region of the α₁ subunit. A fourth subunit, γ, is unique to L-type calcium channels expressed in skeletal muscle T-tubules.

Recently, each of these α₁ subtypes has been cloned and expressed, thus permitting more extensive pharmacological studies. These channels have been designated α_{1A}-α_{1I} and α_{1S} and correlated with the subtypes set forth above. α_{1A} channels are of the P/Q type; α_{1B} represents N; α_{1C}, α′_{1D}, α_{1F} and α_{1S} represent L; α_{1E} represents a novel type of calcium conductance, and α_{1G}-α_{1I} represent members of the T-type family.

Further details concerning the function of N-type channels, which are mainly localized to neurons, have been disclosed, for example, in U.S. Pat. No. 5,623,051, the disclosure of which is incorporated herein by reference. As described, N-type channels possess a site for binding syntaxin, a protein anchored in the presynaptic membrane. Blocking this interaction also blocks the presynaptic response to calcium influx. Thus, compounds that block the interaction between syntaxin and this binding site would be useful in neural protection and analgesia. Such compounds have the added advantage of enhanced specificity for presynaptic calcium channel effects.

U.S. Pat. No. 5,646,149 describes calcium channel antagonists of the formula A-Y-B wherein B contains a piperazine or piperidine ring directly linked to Y. An essential component of these molecules is represented by A, which must be an antioxidant; the piperazine or piperidine itself is said to be important. The exemplified compounds contain a benzhydril substituent, based on known calcium channel blockers (see below). In some cases, the antioxidant can be a phenyl group containing methoxy and/or hydroxyl substituents. In most of the illustrative compounds, however, a benzhydril moiety is coupled to the heterocycle simply through a CH group or C═ group. In the few compounds where there is an alkylene chain between the CH to which the two phenyl groups are bound and the heterocycle, the antioxidant must be coupled to the heterocycle through an unsubstituted alkylene and in most of these cases the antioxidant is a bicyclic system. Where the antioxidant can simply be a phenyl moiety coupled through an alkynylene, the linker from the heterocycle to the phenyl moieties contains no more than six atoms in the chain. U.S. Pat. No. 5,703,071 discloses compounds said to be useful in treating ischemic diseases. A mandatory portion of the molecule is a tropolone residue; among the substituents permitted are piperazine derivatives, including their benzhydril derivatives. U.S. Pat. No. 5,428,038 discloses compounds which are said to exert a neural protective and antiallergic effect. These compounds are coumarin derivatives which may include derivatives of piperazine and other six-membered heterocycles. A permitted substituent on the heterocycle is diphenylhydroxymethyl. Thus, approaches in the art for various indications which may involve calcium channel blocking activity have employed compounds which incidentally contain piperidine or piperazine moieties substituted with benzhydril but mandate additional substituents to maintain functionality.

Certain compounds containing both benzhydril moieties and piperidine or piperazine are known to be calcium channel antagonists and neuroleptic drugs. For example, Gould, R. J. et al. Proc Natl Acad Sci USA (1983) 80:5122-5125 describes antischizophrenic neuroleptic drugs such as lidoflazine, fluspirilene, pimozide, clopimozide, and penfluridol. It has also been shown that fluspirilene binds to sites on L-type calcium channels (King, V. K. et al. J Biol Chem (1989) 264:5633-5641) as well as blocking N-type calcium current (Grantham, C. J. et al. Brit J Pharmacol (1944) 111:483-488). In addition, Lomerizine, as developed by Kanebo K K, is a known calcium channel blocker; Lomerizine is, however, not specific for N-type channels. A review of publications concerning Lomerizine is found in Dooley, D., Current Opinion in CPNS Investigational Drugs (1999)1:116-125.

In addition, benzhydril derivatives of piperidine and piperazine are described in PCT publication WO 00/01375 published Jan. 13, 2000 and incorporated herein by reference. Reference to this type of compound as known in the prior art is also made in WO 00/18402 published Apr. 6, 2000 and in Chiarini, A., et al., Bioorganic and Medicinal Chemistry, (1996) 4:1629-1635.

Various other piperidine or piperazine derivatives containing aryl substituents linked through nonaromatic linkers are described as calcium channel blockers in U.S. Pat. No. 5,292,726; WO 99/43658; Breitenbucher, J. G., et al., Tet Lett (1998) 39:1295-1298.

Certain of the compounds included in the genus described herein have been disclosed to be useful in other contexts. For example, U.S. Pat. No. 3,957,812 describes 2-phenoxyacetamido-5-nitrothiazole compounds which have antibacterial, antifungal and antiparasite activity. Other members of the genus disclosed herein are new compounds.

The present invention is based on the recognition that the combination of a 5-membered heterocyclic ring containing at least one nitrogen and/or at least one sulfur coupled through a linker to a benzhydril or phenyl moiety or their heteroaryl counterparts results in effective calcium channel blocking activity. In some cases enhanced specificity for N-type channels, or T-type channels or decreased specificity for L-type channels is shown. The compounds are useful for treating stroke and pain and other calcium channel-associated disorders, as further described below. By focusing on these moieties, compounds useful in treating indications associated with abnormal calcium channel activity are prepared.

### DISCLOSURE OF THE INVENTION

The invention relates to compounds useful in treating conditions such as stroke, head trauma, migraine, chronic, neuropathic and acute pain, epilepsy, hypertension, cardiac arrhythmias, and other indications associated with calcium metabolism, including synaptic calcium channel-mediated functions. In one aspect, the invention is directed to therapeutic methods that employ compounds of the formula

Ar—linker—Het (1)

or

Ar₂CH—linker—Het (2)

or the salts thereof,

wherein each Ar is independently a 6-membered optionally substituted aromatic ring containing one or more heteroatoms selected from the group consisting of S, O and N, which ring is optionally coupled through —O— to the linker;

the linker is an alkylene type chain of 2-10 sequentially connected atoms selected from the group consisting of C, N, O, and S which connecting atoms are optionally substituted; and

each Het is a 5-membered optionally substituted heterocyclic ring which contains at least one N or S atom.

The Ar and Het rings may also optionally be substituted. Preferred substituents on Ar, the connecting atoms of the linker, and Het include optionally substituted alkyl (1-6C), optionally substituted alkenyl (2-6C), optionally substituted alkynyl (2-6C), halo, CN, CF₃, OCF₃, OCF, NO₂, NR₂, OR, SR, COR, COOR, CONR₂, NROCR and OOCR where R is H or alkyl (1-6C) and may also include an aryl substituent, wherein two substituents may form a 5-7 membered ring, and each R also optionally being unsaturated and/or having one C replaced by one or more heteroatoms selected from O, N and S. The alkyl, alkenyl, and alkynyl groups may also contain one or more heteroatoms.

The substituents on alkyl, alkenyl, and alkynyl are similar to those set forth above and may further include, for example, ═O.

The invention is directed to methods to antagonize calcium channel activity using the compounds of formulas (1) and (2) and thus to treat associated conditions. It will be noted that these conditions are associated with abnormal calcium channel activity. In another aspect, the invention is directed to pharmaceutical compositions containing these compounds. The invention is also directed to certain novel compounds.

### BRIEF DESCRIPTION OF THE DRAWINGS

FIG. 1 shows preferred compounds of the invention.

FIGS. 2A, 2B, and 2C show the ability of one compound of the invention, 79-B-8 shown on page 1 of FIG. 1, to block various calcium ion channels.

FIGS. 3A and 3B show similar results for an additional compound of the invention, NT044, shown on page 4 of FIG. 1.

FIGS. 4A and 4B show similar results for an additional compound of the invention, NT051, shown on page 3 of FIG. 1.

### MODES OF CARRYING OUT THE INVENTION

The compounds of formulas (1) and (2) useful in the methods of the invention, exert their desirable effects through their ability to antagonize the activity of calcium channels. This makes them useful for treatment of certain conditions. Among such conditions are stroke, epilepsy, head trauma, migraine and chronic, neuropathic and acute pain. Calcium flux is also implicated in other neurological disorders such as schizophrenia, anxiety, depression, other psychoses, and certain degenerative disorders. Other treatable conditions include cardiovascular conditions such as hypertension and cardiac arrhythmias.

While the compounds of formulas (1) and (2) generally have this activity, the availability of a multiplicity of calcium channel blockers permits a nuanced selection of compounds for particular disorders. Thus, the availability of this class of compounds provides not only a genus of general utility in indications that are affected by excessive calcium channel activity, but also provides a large number of compounds which can be mined and manipulated for specific interaction with particular forms of calcium channels.

The availability of recombinantly produced calcium channels of the α_{1A}-α_{1I} and α_{1S} types set forth above, facilitates this selection process. Dubel, S. J. et al. Proc Natl Acad Sci USA (1992) 89:5058-5062; Fujita, Y. et al. Neuron (1993) 10:585-598; Mikami, A. et al. Nature (1989) 340:230-233; Mori, Y. et al. Nature (1991) 350:398-402; Snutch, T. P. et al. Neuron (1991) 7:45-57; Soong, T. W. et al. Science (1993) 260:1133-1136; Tomlinson, W. J. et al. Neuropharmacology (1993) 32:1117-1126; Williams, M. E. et al. Neuron (1992) 8:71-84; Williams, M. E. et al. Science (1992) 257:389-395; Perez-Reyes, et al. Nature (1998) 391:896-900; Cribbs, L. L. et al. Circulation Research (1998) 83:103-109; Lee, J. H. et al. Journal of Neuroscience (1999) 19:1912-1921.

Thus, while it is known that calcium channel activity is involved in a multiplicity of disorders, the types of channels associated with particular conditions is the subject of ongoing data collection. For example, the association of N-type channels in conditions associated with neural transmission would indicate that compounds of the invention which target N-type receptors are most useful in these conditions. Many of the members of the genus of compounds of formulas (1) and (2) exhibit high affinity for N-type channels; other members of the genus may preferentially target T-type channels.

There are two distinguishable types of calcium channel inhibition. The first, designated “open channel blockage,” is conveniently demonstrated when displayed calcium channels are maintained at an artificially negative resting potential of about −100 mV (as distinguished from the typical endogenous resting maintained potential of about −70 mV). When the displayed channels are abruptly depolarized under these conditions, calcium ions are caused to flow through the channel and exhibit a peak current flow which then decays. Open channel blocking inhibitors diminish the current exhibited at the peak flow and can also accelerate the rate of current decay.

This type of inhibition is distinguished from a second type of block, referred to herein as “inactivation inhibition.” When maintained at less negative resting potentials, such as the physiologically important potential of −70 mV, a certain percentage of the channels may undergo conformational change, rendering them incapable of being activated—i.e., opened—by the abrupt depolarization. Thus, the peak current due to calcium ion flow will be diminished not because the open channel is blocked, but because some of the channels are unavailable for opening (inactivated). “Inactivation” type inhibitors increase the percentage of receptors that are in an inactivated state.

Synthesis

The compounds of the invention may be synthesized using conventional methods. Illustrative of such methods is the following.

O-benzotriazolyl-N,N,N′,N′-tetramethyluronium tetrafluoroborate (1.2 equi.) is added to a solution of the corresponding acid (1 equi.), amine (1 equi.) and triethylamine (0.1 ml) in methylene chloride (10 ml) and acetonitrile (5 ml) and the reaction mixture is stirred at room temperature overnight. If there is precipitate in the reaction mixture, the solid is collected by filtration and washed with methylene chloride. If the reaction mixture remains in solution, solvents are removed by evaporation and the residue dissolved in ethyl acetate (20 ml) and washed with 10% sodium bicarbonate aqueous solution, water, 10% citric acid aqueous solution and brine successively. The ethyl acetate solution is dried over magnesium sulfate. After removal of the drying agent by filtration, the filtrate is concentrated. The residue is applied to flash column chromatography with silica gel (230-400 meshes) and ethyl acetate and hexanes as eluents.

The illustrative method above is appropriate for the synthesis of compounds wherein the linker contains an amide. The, amide can be converted to the reduced form by conventional methods to reduce carbonyl groups.

Preferred Embodiments

The compounds of formulas (1) and (2) are defined as shown in terms of the embodiments of their various substituents.

Preferred embodiments of Ar include phenyl, 2-, 3-, and 4-pyridyl, 2, 6- and 3, 5-pyrimidinyl, each of which may be optionally substituted. Preferably, the phenyl moieties contain 0-3 substituents, more preferably 0-2 substituents; the nitrogen or other heteroatom containing rings preferably contain 0-2 substituents. Preferred substituents on the aryl moieties include halo, optionally substituted alkyl, optionally substituted alkoxy, and optionally substituted alkyl or dialkyl amino. Particularly preferred are unsubstituted alkyl, unsubstituted alkoxy, chloro, bromo and fluoro.

Preferred embodiments of Het include 5-membered rings which contain a single nitrogen, two nitrogens, three nitrogens, a sulfur, a sulfur and one nitrogen, a sulfur and two nitrogens, and the corresponding oxygen containing 5-membered rings. These rings are preferably unsaturated and thus aromatic, but may optionally contain only one pi bond or no pi bonds. These rings may also optionally be substituted, preferably by a single substituent.

Particularly preferred embodiments of Het include thiazole, dihydrothiazole, azothiazole, imidazole, triazines, and the like. Preferred substituents include, for example, halo, NH_{2,} OH, SH, OPO₃H₂, NO₂ and the like as well as optionally substituted and optionally heteroatom containing alkyl (1-6 chain members), alkenyl (2-6 chain members) and alkynyl (2-6 chain members). The heteroatoms contained in the substituents are typically S, O, N or P. Typical substituents may include aryl, arylalkyl, arylalkenyl, ═O, CN, CF₃, OCF₃, OCF, NO₂, NR₂, OR, SR, COR, COOR, CONR₂, NROCR, NOOCR, where R is alkyl (1-6C), and may include an aryl substituent.

Particularly preferred linkers are those which contain amides, in particular wherein the amide is directly bound to the heterocycle, Het. Also preferred are linkers which contain oxygen as a heteroatom instead of or in addition to the amide linkage. Preferred linkers contain 4-6 members in the directly linking chain.

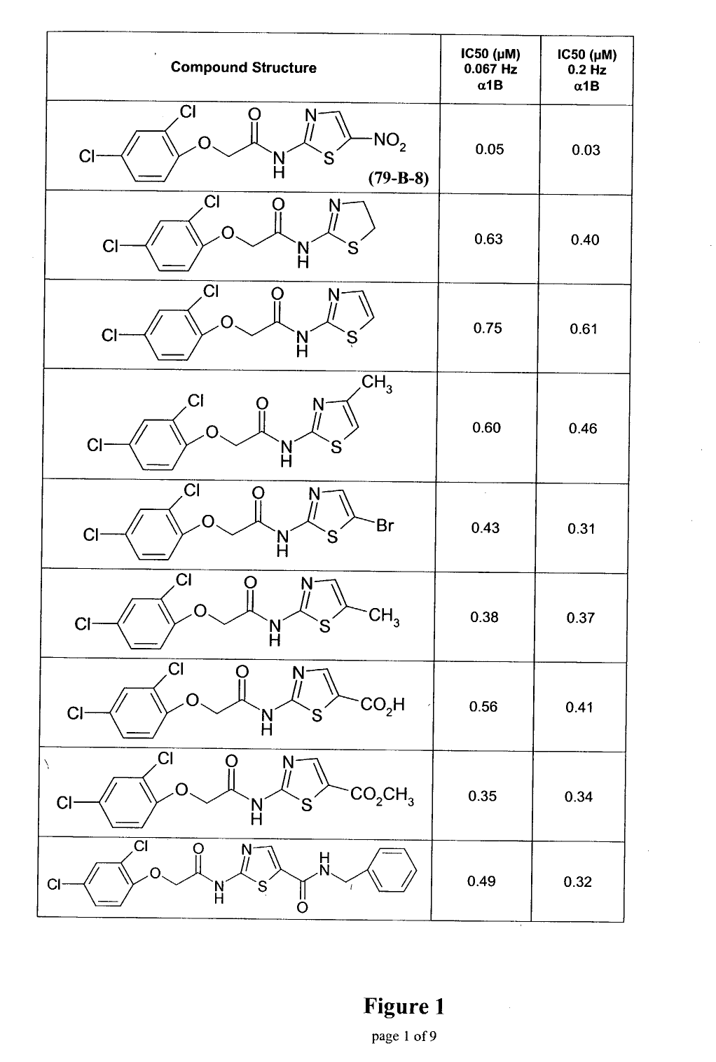

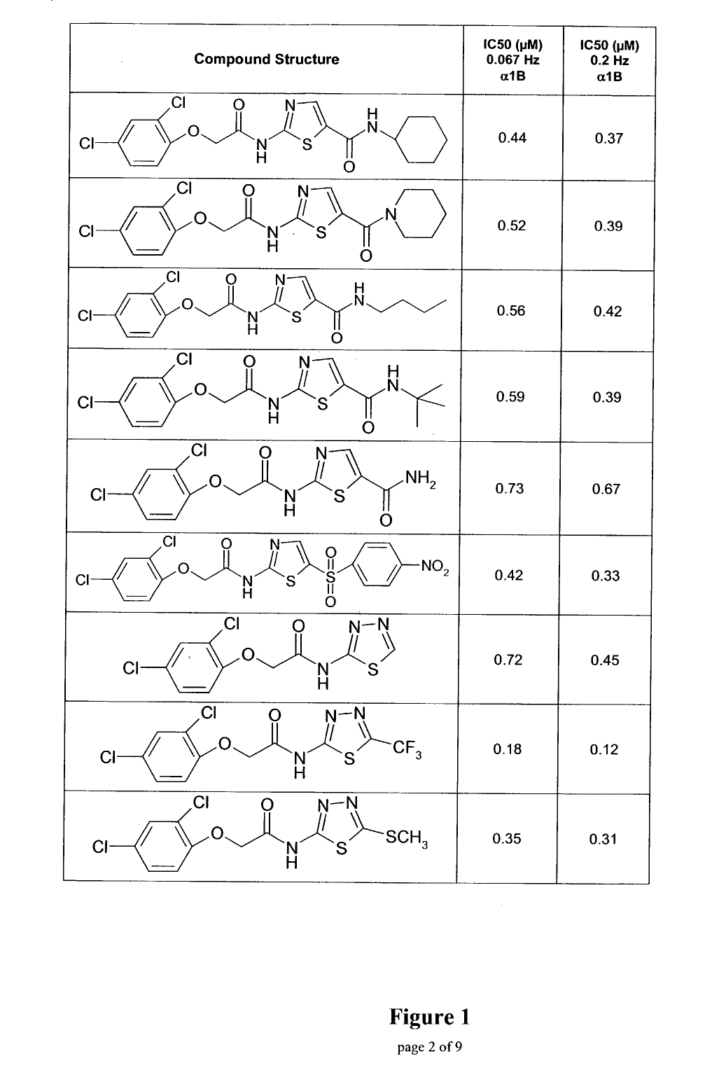

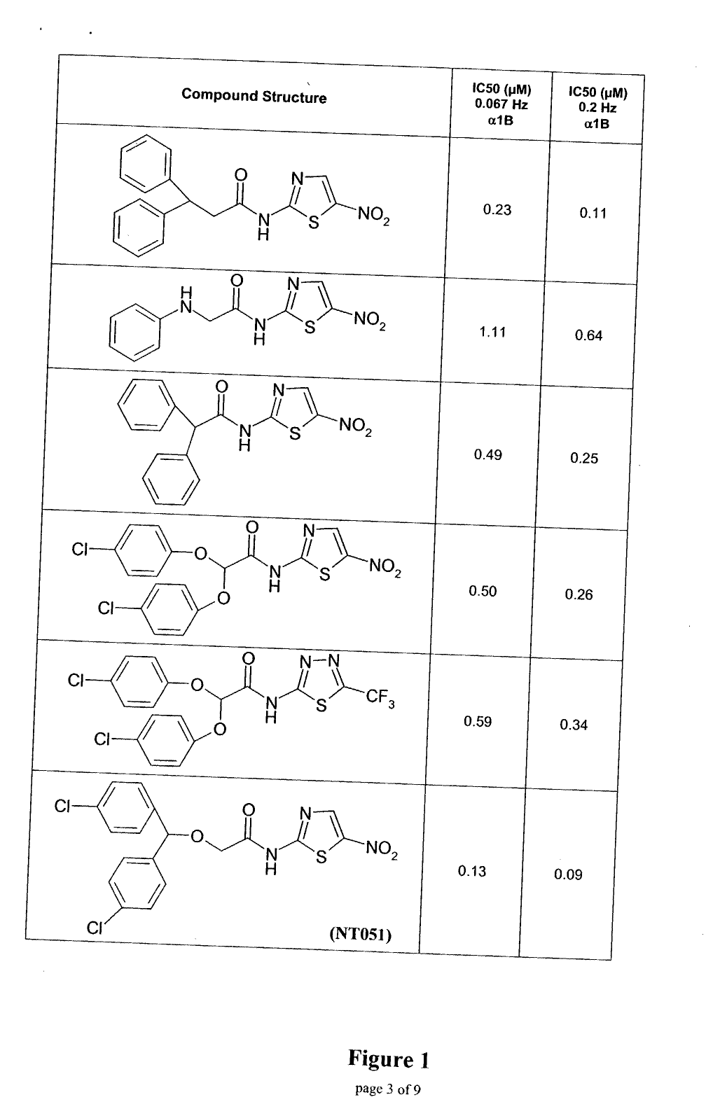

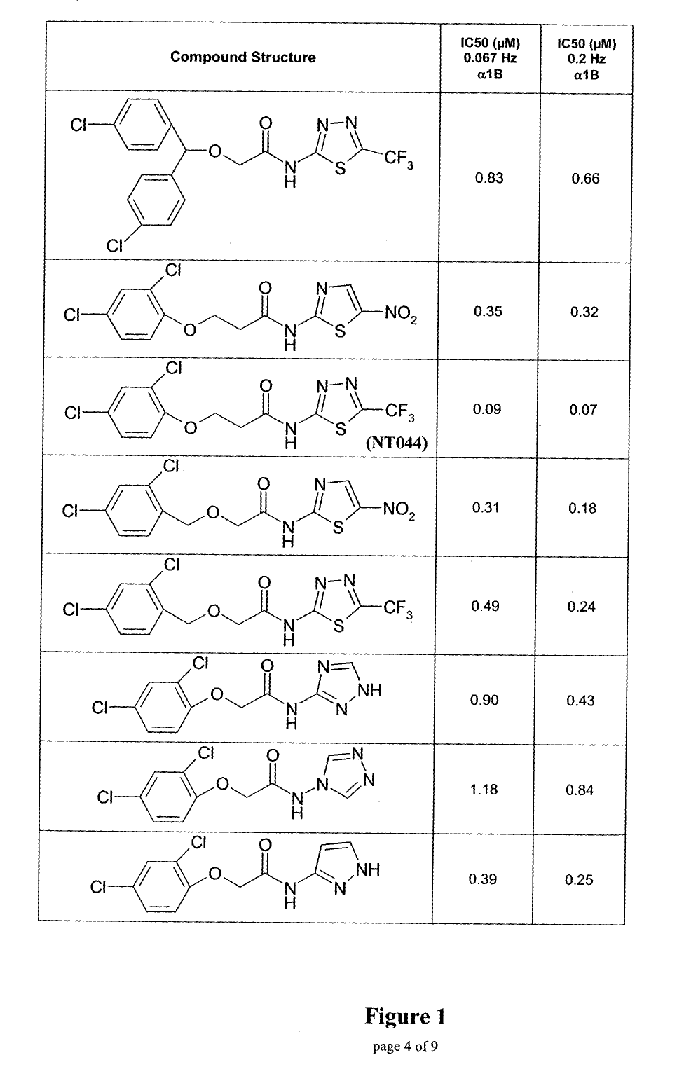

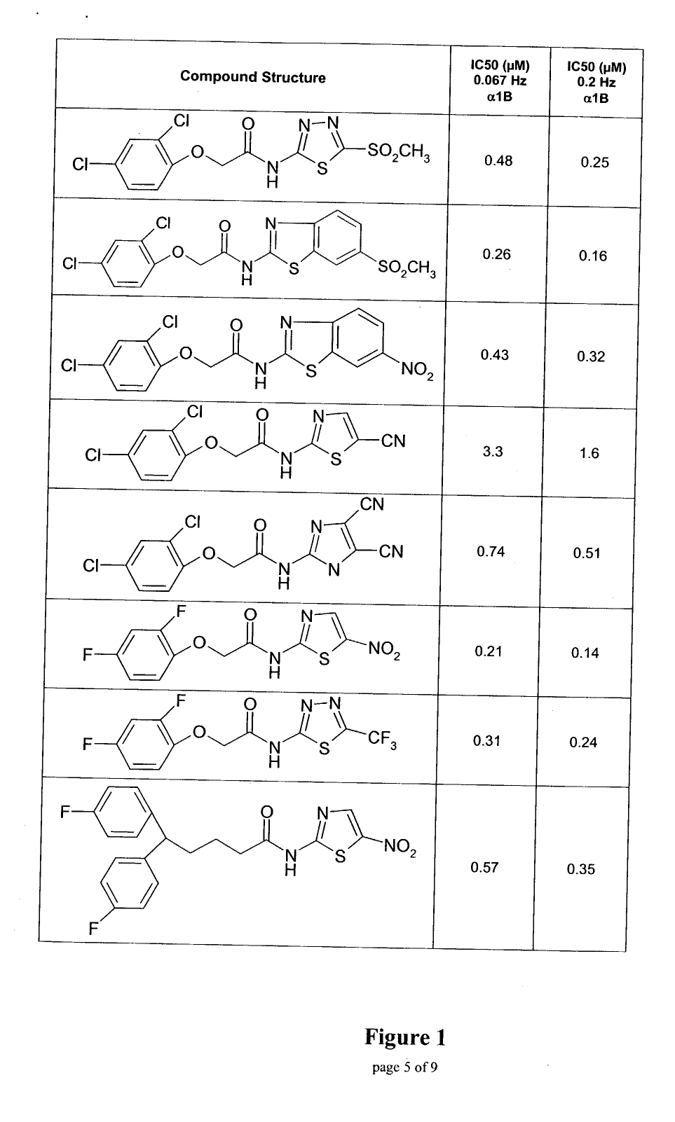

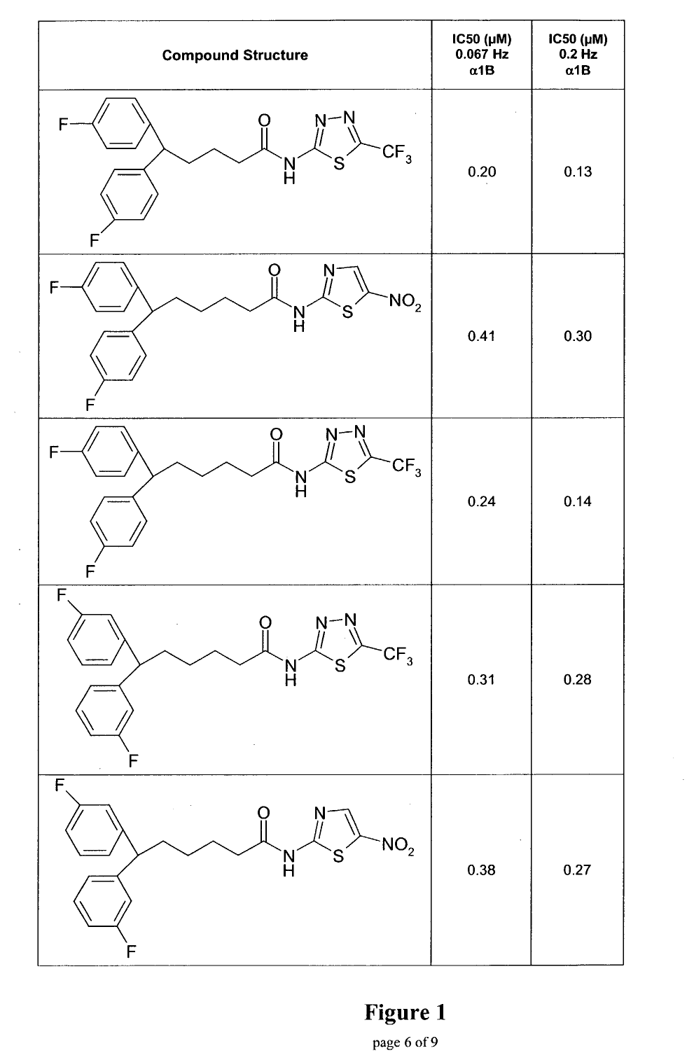

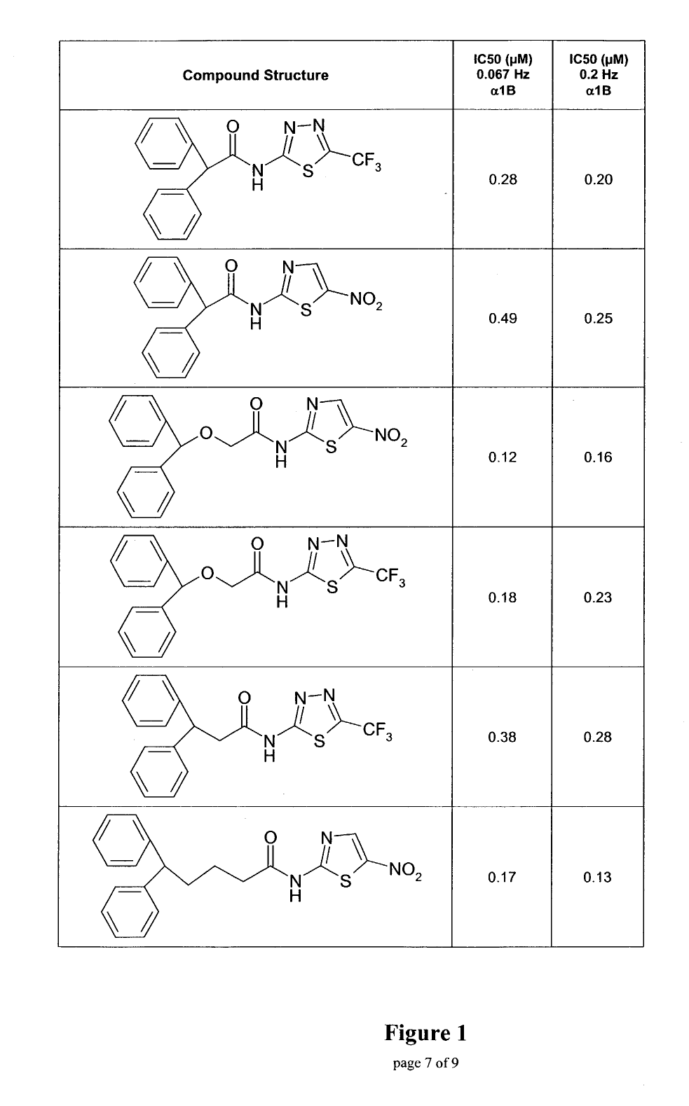

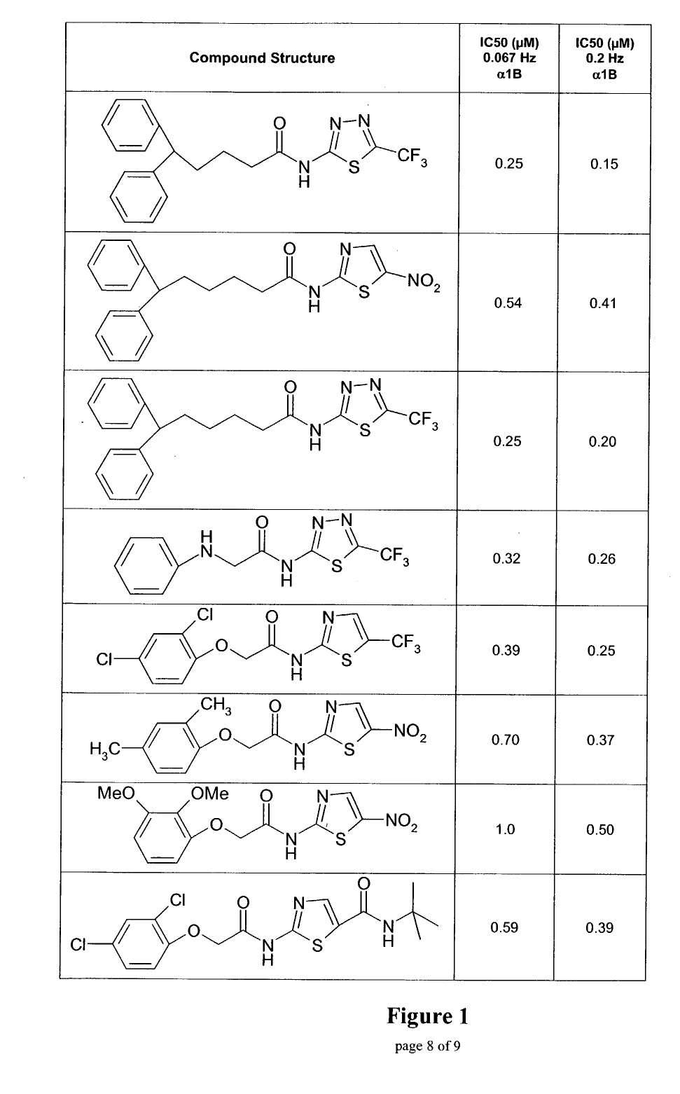

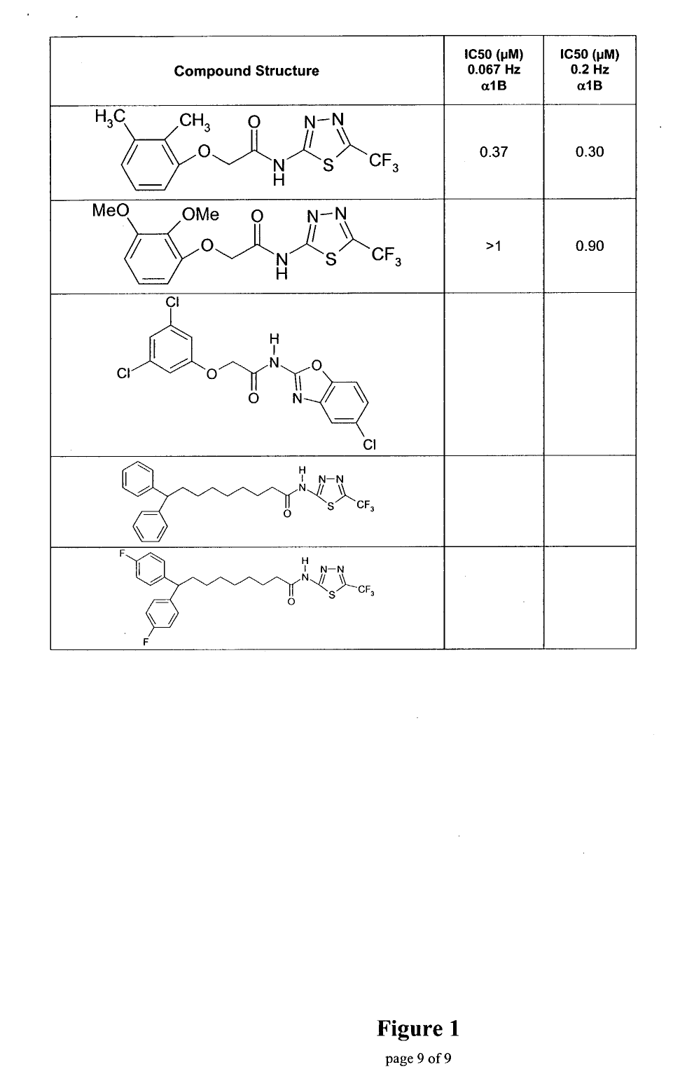

Particularly preferred compounds are those set forth in FIG. 1.

Preferred embodiments of Het include the following:

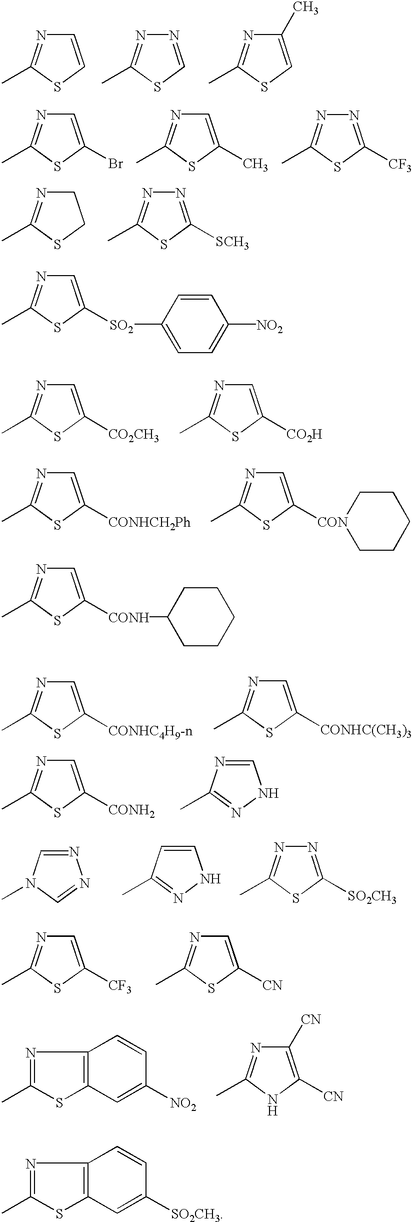

Particularly preferred substituents on Ar include halo, especially Cl and F, alkyl (1-6C) and alkoxy (1-6C).

The “linker” contains 2-10 contiguous atoms which form a single chain linking Ar (or in the case of formula (2), CH) with Het. Preferred linkers include (CH₂)ₙCONH and (CH₂)ₙ₊₁NH where n is 0-8. It will be noted that each Ar may optionally be coupled to the linker through an oxygen atom—i.e., the Ar and linker are participants in an ether bond. Several of the structures shown in FIG. 1 have this feature.

Libraries and Screening

The compounds of the invention can be synthesized individually using methods known in the art per se, or as members of a combinatorial library.

Synthesis of combinatorial libraries is now commonplace in the art. Suitable descriptions of such syntheses are found, for example, in Wentworth, Jr., P. et al. Current Opinion in Biol (1993) 9:109-115; Salemme, F. R. et al. Structure (1997) 5:319-324. The libraries contain compounds with various substitutents and various degrees of unsaturation, as well as different chain lengths. The libraries, which contain, as few as 10, but typically several hundred members to several thousand members, may then be screened for compounds which are particularly effective against a specific subtype of calcium channel, i.e., the N-type channel. In addition, using standard screening protocols, the libraries may be screened for compounds which block additional channels or receptors such as sodium channels, potassium channels and the like.

Methods of performing these screening functions are well known in the art. Typically, the receptor to be targeted is expressed at the surface of a recombinant host cell such as human embryonic kidney cells. The ability of the members of the library to bind the channel to be tested is measured, for example, by the ability of the compound in the library to displace a labeled binding ligand such as the ligand normally associated with the channel or an antibody to the channel. More typically, ability to antagonize the receptor is measured in the presence of calcium ion and the ability of the compound to interfere with the signal generated is measured using standard techniques.

In more detail, one method involves the binding of radiolabeled agents that interact with the calcium channel and subsequent analysis of equilibrium binding measurements including, but not limited to, on rates, off rates, K_{d} values and competitive binding by other molecules. Another method involves the screening for the effects of compounds by electrophysiological assay whereby individual cells are impaled with a microelectrode and currents through the calcium channel are recorded before and after application of the compound of interest. Another method, high-throughput spectrophotometric assay, utilizes loading of the cell lines with a fluorescent dye sensitive to intracellular calcium concentration and subsequent examination of the effects of compounds on the ability of depolarization by potassium chloride or other means to alter intracellular calcium levels.

As described above, a more definitive assay can be used to distinguish inhibitors of calcium flow which operate as open channel blockers, as opposed to those that operate by promoting inactivation of the channel. The methods to distinguish these types of inhibition are more particularly described in the examples below. In general, open-channel blockers are assessed by measuring the level of peak current when depolarization is imposed on a background resting potential of about −100 mV in the presence and absence of the candidate compound. Successful open-channel blockers will reduce the peak current observed and may accelerate the decay of this current. Compounds that are inactivated channel blockers are generally determined by their ability to shift the voltage dependence of inactivation towards more negative potentials. This is also reflected in their ability to reduce peak currents at more depolarized holding potentials (e.g., −70 mV) and at higher frequencies of stimulation, e.g., 0.2 Hz vs. 0.03 Hz.

Utility and Administration

For use as treatment of human and animal subjects, the compounds of the invention can be formulated as pharmaceutical or veterinary compositions. Depending on the subject to be treated, the mode of administration, and the type of treatment desired—e.g., prevention, prophylaxis, therapy; the compounds are formulated in ways consonant with these parameters. A summary of such techniques is found in Remington's Pharmaceutical Sciences, latest edition, Mack Publishing Co., Easton, Pa., incorporated herein by reference.

In general, for use in treatment, the compounds of formula (1) or (2) may be used alone, as mixtures of two or more compounds of formula (1) or (2) or in combination with other pharmaceuticals. Depending on the mode of administration, the compounds will be formulated into suitable compositions to permit facile delivery.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, preservatives and the like. The compounds can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, as in understood in the art.

For administration to animal or human subjects, the dosage of the compounds of the invention is typically 0.1-15 mg/kg, preferably 0.1-1 mg/kg. However, dosage levels are highly dependent on the nature of the condition, the condition of the patient, the judgment of the practitioner, and the frequency and mode of administration.

The following examples are intended to illustrate but not to limit the invention.

### EXAMPLE 1

### Assessment of Calcium Channel Blocking Activity

Antagonist activity was measured using whole cell patch recordings on human embryonic kidney cells either stably or transiently expressing rat α_{1B}+α_{2b}+β_{1b} channels (N-type channels) with 5 mM barium as a charge carrier.

For transient expression, host cells, such as human embryonic kidney cells, HEK 293 (ATCC# CRL 1573) were grown in standard DMEM medium supplemented with 2 mM glutamine and 10% fetal bovine serum. HEK 293 cells were transfected by a standard calcium-phosphate-DNA coprecipitation method using the rat α_{1B}+β_{1b}+α₂δ N-type calcium channel subunits in a vertebrate expression vector (for example, see Current Protocols in Molecular Biology).

After an incubation period of from 24 to 72 hrs the culture medium was removed and replaced with external recording solution (see below). Whole cell patch clamp experiments were performed using an Axopatch 200B amplifier (Axon Instruments, Burlingame, Calif.) linked to an IBM compatible personal computer equipped with pCLAMP software. Borosilicate glass patch pipettes (Sutter Instrument Co., Novato, Calif.) were polished (Microforge, Narishige, Japan) to a resistance of about 4 MΩ when filled with cesium methanesulfonate internal solution (composition in MM: 109 CsCH₃SO₄, 4 MgCl₂, 9 EGTA, 9 HEPES, pH 7.2). Cells were bathed in 5 mM Ba⁺⁺ (in mM: 5 BaCl₂, 1 MgCl₂, 10 HEPES, 40 tetraethylammonium chloride, 10 glucose, 87.5 CsCl pH 7.2). Current data shown were elicited by a train of 100 ms test pulses at 0.066 Hz from −100 mV and/or −80 mV to various potentials (min. −20 mV, max. +30 mV). Drugs were perfused directly into the vicinity of the cells using a microperfusion system.

Normalized dose-response curves were fit (Sigmaplot 4.0, SPSS Inc., Chicago, Ill.) by the Hill equation to determine IC₅₀ values. Steady-state inactivation curves were plotted as the normalized test pulse amplitude following 5 s inactivating prepulses at +10 mV increments. Inactivation curves were fit (Sigmaplot 4.0) with the Boltzman equation, Iₚₑₐₖ (normalized)=1/(1+exp((V−Vₕ)z/25.6)), where V and Vₕ are the conditioning and half inactivation potentials, respectively, and z is the slope factor.

### EXAMPLE 2

### Additional Methods and L and P/Q Channel Types

The method of Example 1 was followed with slight modifications as will be apparent from the description below.

A. Transformation of HEK Cells:

N-type calcium channel blocking activity was assayed in human embryonic kidney cells, HEK 293, stably transfected with the rat brain N-type calcium channel subunits (α_{1B}+α_{2δ}+β_{1b} cDNA subunits). Alternatively, N-type calcium channels (α_{1B}+α_{2δ}+β_{1b} cDNA subunits), L-type channels (α_{1C}+α_{2δ}+β_{1b} cDNA subunits) and P/Q-type channels (α_{1A}+α_{2δ}+β_{1b} cDNA subunits) were transiently expressed in HEK 293 cells. Briefly, cells were cultured in Dulbecco's modified eagle medium (DMEM) supplemented with 10% fetal bovine serum, 200 U/ml penicillin and 0.2 mg/ml streptomycin at 37° C. with 5% CO₂. At 85% confluency cells were split with 0.25% trypsin/1 mM EDTA and plated at 10% confluency on glass coverslips. At 12 hours the medium was replaced and the cells transiently transfected using a standard calcium phosphate protocol and the appropriate calcium channel cDNAs. Fresh DMEM was supplied and the cells transferred to 28° C./ 5% CO₂. Cells were incubated for 1 to 2 days to whole cell recording.

B. Measurement of Inhibition:

Whole cell patch clamp experiments were performed using an Axopatch 200B amplifier (Axon Instruments, Burlingame, Calif.) linked to a personal computer equipped with pCLAMP software. The external and internal recording solutions contained, respectively, 5 mM BaCl₂, 1 mM MgCl₂, 10 mM HEPES, 40 mM TEACl, 10 mM glucose, 87.5 mM CsCl (pH 7.2) and 108 mM CsMS, 4 mM MgCl₂, 9 mM EGTA, 9 mM HEPES (pH 7.2). Currents were typically elicited from a holding potential of −80 mV to +10 mV using Clampex software (Axon Instruments). Typically, currents were first elicited with low frequency stimulation (0.03 Hz) and allowed to stabilize prior to application of the compounds. The compounds were then applied during the low frequency pulse trains for two to three minutes to assess tonic block, and subsequently the pulse frequency was increased to 0.2 Hz to assess frequency dependent block. Data were analyzed using Clampfit (Axon Instruments) and SigmaPlot 4.0 (Jandel Scientific).

### EXAMPLE 3

### Assay for T-Type Channel Blockage

Cell lines (HEK 293) stably expressing α_{1G} are employed (passage number 10-25). The standard whole-cell patch-clamp technique is used (AXOPATCH 200B and CLAMPEX 7 software package). The external solution contains 132 mM CsCl, 2 mM CaCl₂, 1 mM MgCl₂, 10 mM HEPES, and 10 mM glucose and is brought to pH 7.4 with CsOH. The tonicity is 265.5 mOsm. The internal solution contains 108 mM Cs-methanesulfonate; 2 mM MgCl₂, 10 mM HEPES, 11 mM EGTA-Cs, 2 mM ATP, and is brought to pH 7.3 with CsOH and has a tonicity of 270 mOsm.

To fully activate the T-type inward calcium current, short command steps to −40 mV is applied every 15 seconds from a holding potential of −100 mV. To study partially inactivated T-type currents, 10 second pulses to −75 mV or −80 mV are used. Test compounds are diluted daily at 100 nM, with final DMSO at 0.01% (v/v), from 1 mM DMSO stock aliquots. The solutions are applied via a fine tubing positioned near the cell.

### EXAMPLE 4

### Synthesis of NT-040

O-benzotriazolyl-N,N,N′,N′-tetramethyluronium tetrafluoroborate (0.187 g, 0.58 mmol) is added to a solution of bis-(4-chloro-phenoxy) acetic acid (0.164 g, 0.5 mmol), 2-amino-5-trifluoromethyl- 1,3,4-thiadiazole (0.089 g, 0.5 mmol) and triethylamine (0.1 ml) in methylene chloride (10 ml) and acetonitrile (5 ml) and the reaction mixture is stirred at room temperature overnight. The solvents are removed by evaporation and the residue is dissolved in ethyl acetate (20 ml) and washed with 10% sodium bicarbonate aqueous solution, water, 10% citric acid aqueous solution and brine successively. The ethyl acetate solution is dried over magnesium sulfate. After removal of the drying agent by filtration, the filtrate is concentrated. The residue is applied to flash column chromatography with silica gel (230-400 meshes) and ethyl acetate and hexanes (1:5) as eluents. Yield: 81%.

### EXAMPLE 5

### Synthesis of NM 198

O-benzotriazolyl-N,N,N′,N′-tetramethyluronium tetrafluoroborate (0.38 g, 1.2 mmol) is added to a solution of 2[2-(2,4-dichlorophenoxy)-acetylamino)-thiazole-5-carboxylic acid (0.347 g, 1 mmol), cyclohexylamine (0.10 g, 1 mmol) and triethylamine (0.16 ml) in methylene chloride (10 ml) and acetonitrile (5 ml) and the reaction mixture is stirred at room temperature overnight. The resulting suspension is filtered and the collected solid is washed with excessive amount of methylene chloride. Yield: 86%.

### EXAMPLE 6

### Synthesis of NT 044

Synthesis of 3-(2,4-dichlorophenoxy) propionic acid

Sodium hydride (2g, 50 mmol, 60% dispersed in mineral oil) suspended in anhydrous THF (30 ml) cooled at 0° C. flushed with nitrogen was added dropwise a solution of 2,4-dichlorobphenol (2.47 g, 15 mmol) in anhydrous THF (15 ml). 2-Bromopropionic acid (2.84 g, 15 mmol) in anhydrous THF (15 ml) was added dropwise and the reaction mixture was refluxed for 7 hours. THF was removed by evaporation. The residue was dissolved in water (50 ml) and the aqueous solution was extracted with chloroform (50 ml×2). The organic solution was discarded. The aqueous solution was then acidified with 6M hydrochloric acid and extracted with chloroform (50 ml×2). The combined organic solution was washed with brine and dried over magnesium sulfate for 3 hours. The drying agent was filtered and the filtrate was concentrated. The residue was applied to flash column chromatography with silica gel (230-400 meshes) and ethyl acetate and hexanes (1:3) as eluents. Yield: 12.5%.

Synthesis of N-2- (5-trifluoromethyl-1,3,4-thiadiazolyl)-3-(2,4-dichlorophenoxy) propionyl amide

O-benzotriazolyl-N,N,N′,N′-tetramethyluronium tetrafluoroborate (0,187 g, 0.58 mmol) was added to a solution of 3-(2,4-dichlorophenoxy) propionic acid (0.12 g, 0.5 mmol), 2-amino-5-trifluoromethyl-1,3,4,-thiadiazole (0.087 g, 0.5 mmol) and triethylamine (0.1 ml) in methylene chloride (10 ml) and acetonitrile (5 ml) and the reaction mixture was stirred at room temperature overnight. The solvents were removed by evaporation and the residue was dissolved in ethyl acetate (20 ml) and washed with 10% sodium bicarbonate aqueous solution, water, 10% citric acid aqueous solution and brine successively. The ethyl acetate solution was dried over magnesium sulfate. After removal of the drying agent by filtration, the filtrate was concentrated. The residue was applied to flash column chromatography with silica gel (230-400 meshes) and ethyl acetate and hexanes (1:4) as eluents. Yield: 3.1%.

### EXAMPLE 7

### Preparation of NT 051

Synthesis of 2-(4,4′-dichlorobenzhydryl) acetic acid

Sodium hydride (1.25 g, 31.25 mmol, 60% dispersed in mineral oil) suspended in anhydrous THF (30 ml) cooled at 0° C. flushed with nitrogen was added dropwise a solution of 4,4′-dichlorobenzhydrol (2.58 g, 10 mmol) in anhydrous THF (15 ml). 2-Bromoacetic acid (1.39 g, 10 mmol) in anhydrous THF (15 ml) was added dropwise and the reaction mixture was refluxed for 7 hours. THF was removed by evaporation. The residue was dissolved in water (50 ml) and the aqueous solution was extracted with chloroform (50 ml×2). The organic solution was discarded. The aqueous solution was then acidified with 6M hydrochloric acid and extracted with chloroform (50 ml×2). The combined organic solution was washed with brine and dried over magnesium sulfate for 3 hours. The drying agent was filtered and the filtrate was concentrated. The residue was applied to flash column chromatography with silica gel (230-400 meshes) and ethyl acetate and hexanes (1:3) as eluents. Yield: 72%.

Synthesis of N-2-(5-nitro-thiazolyl)-2-(4,4′-dichlorobenzhydryl) acetic amide

O-benzotriazolyl-N,N,N′,N′-tetramethyluronium tetrafluoroborate (0,187 g, 0.58 mmol) was added to a solution of 2-(4,4′-dichlorobenzhydryl) acetic acid (0.156 g, 0.5 mmol), 2-amino-5-nitro-thiazole (0.075 g, 0.5 mmol) and triethylamine (0.1 ml) in methylene chloride (10 ml) and acetonitrile (5 ml) and the reaction mixture was stirred at room temperature overnight. The solvents were removed by evaporation and the residue was dissolved in ethyl acetate (20 ml) and washed with 10% sodium bicarbonate aqueous solution, water, 10% citric acid aqueous solution and brine successively. The ethyl acetate solution was dried over magnesium sulfate. After removal of the drying agent by filtration, the filtrate was concentrated. The residue was applied to flash column chromatography with silica gel (230-400 meshes) and ethyl acetate and hexanes (1:7) as eluents. Yield: 81%.

### EXAMPLE 8

### Channel Blocking Activities of Various Invention Compounds

Using the procedure set forth in Example 1, various compounds of the invention were tested for their ability to block N-type calcium channels. The results are shown in FIG. 1. The IC₅₀ values are reported in μM.

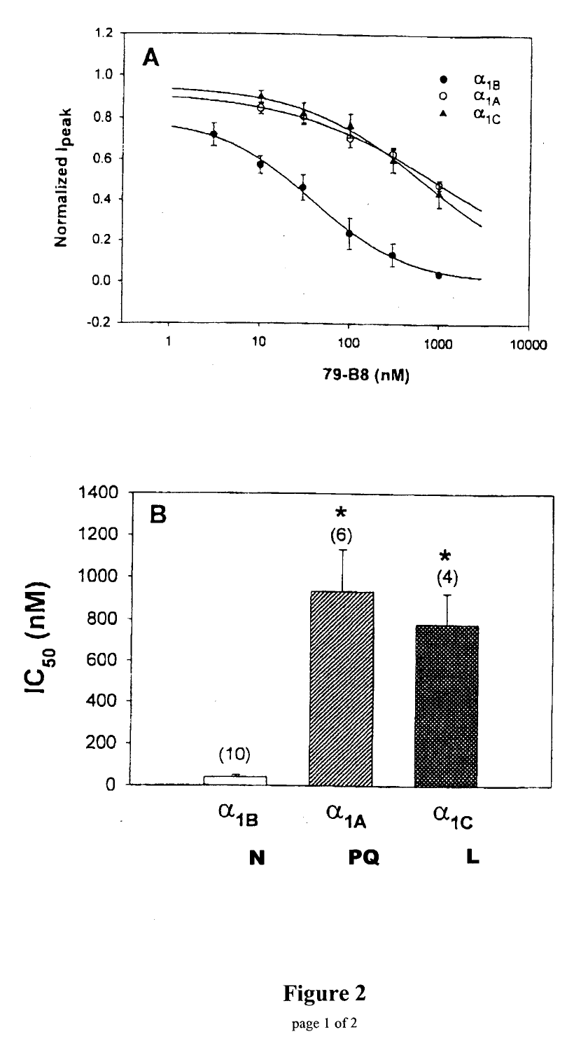

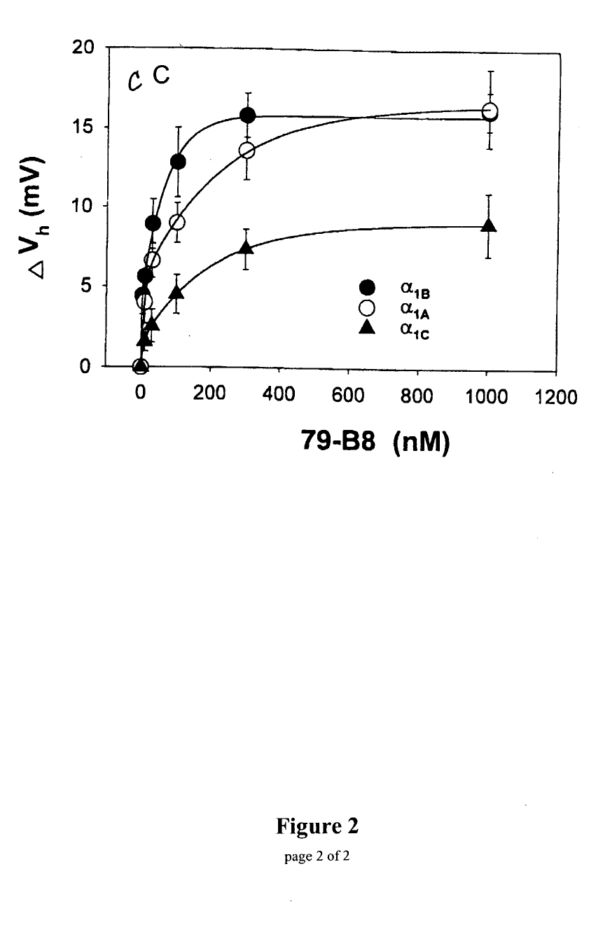

Various compounds were also tested according to the procedure in Example 2 for their ability to inhibit N-type (α_{1B}) P/Q-type (α_{1A}) and L-type (α_{1C}). FIGS. 2A, 2B and 2C show the results for a commercially available compound, shown in FIG. 1, page 1, as compound 79-B8.

FIG. 2A shows a dose response curve for 79-B8 on these channels; FIG. 2B shows a graphical representation of the results calculated as IC₅₀ in nM, and FIG. 2C shows the dose dependent of the shift in half-inactivation voltage of the steady state inactivation to the hyperpolarized direct ion by compound 79-B8.

Based on these results, the estimated IC₅₀ for the N-type channel is 0.039 μM at peak current amplitude and 0.033 , =M at the half-inactivation voltage at steady state. Comparable values for the P/Q channel are 0.94 μM and 0.12 μM, respectively, and for the L-type channel 0.78 μM and 0.10 μM, respectively. This results in N:P ratios at these voltages of 24 and 3.5 and N:L ratios at these voltages at 20 and 3.1, respectively.

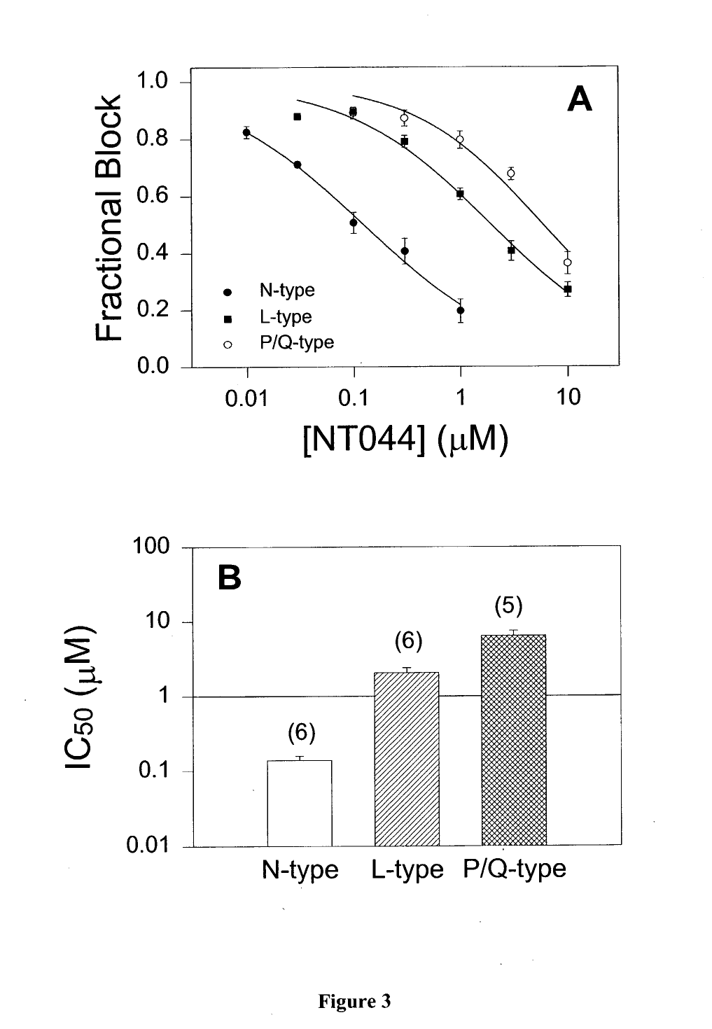

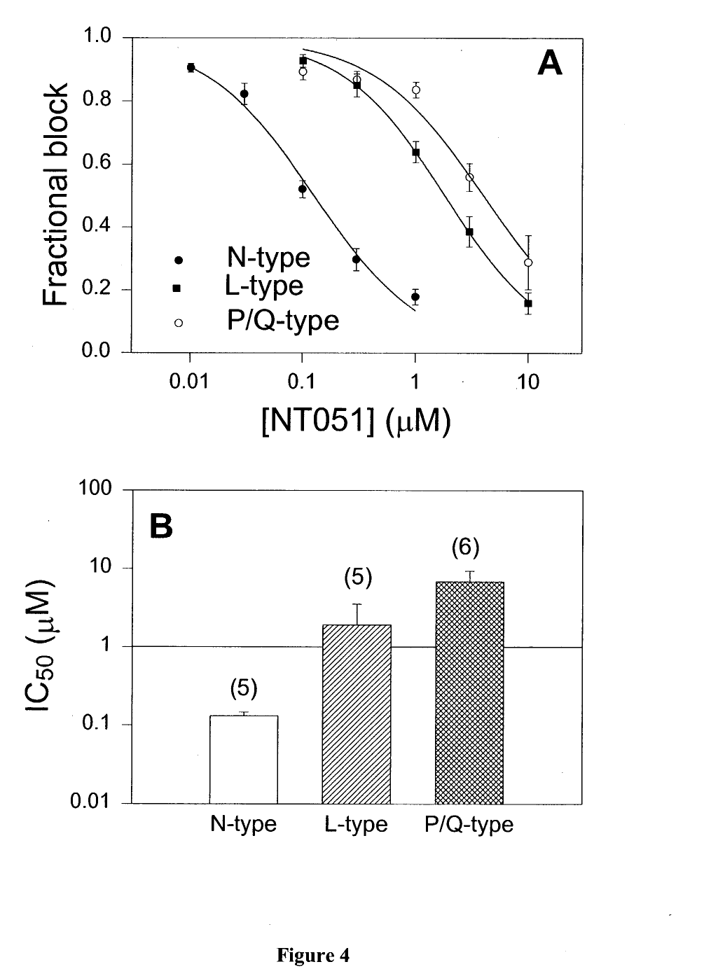

FIGS. 3A and 3B and 4A and 4B show analogous results for compounds shown in FIG. 1 pages 4 and 3 as NT 044 and NT 051, respectively. FIGS. 3A and 4A show fractional block curves as a function of concentration for the three N-type, L-type and P/Q-type channels and FIGS. 3B and 4B show graphical depictions of the calculated IC₅₀'s for the various types of channel. As seen, both NT 044 and NT 051 are somewhat selective for N-type channels as is 79-B8. These results are charted in Table 1.

TABLE 1

| | IC₅₀ | | | | |
|---|---|---|---|---|---|
| | (μM) at 0.1 Hz | | | Ratios | |
| | α_{1B} | α_{1A} | α_{1C} | N:P | N:L |
| NT044 | 0.14 | 6.4 | 2.06 | 45.7:1 | 14.7:1 |
| NT051 | 0.13 | 6.8 | 1.91 | 52.3:1 | 14.7:1 |

All recordings were conducted by a step voltage of +20 mV from a holding potential of −80 mV, at a stimulus frequency of 0.1 H, in 5 mM barium external solution.

1. A method to treat conditions associated with abnormal calcium ion channel activity which method comprises administering to a subject in need of such treatment an effective amount of a compound of the formula
Ar—linker—Het (1)
or
Ar₂CH—linker—Het (2)
or the salts thereof,
wherein each Ar is independently a 6-membered optionally substituted aromatic ring containing one or more heteroatoms selected from the group consisting of S, O and N, which ring is optionally coupled through —O— to the linker;
the linker is an alkylene type chain of 2-10 sequentially connected atoms selected from the group consisting of C, N, O, and S which connecting atoms are optionally substituted; and
each Het is a 5-membered optionally substituted heterocyclic ring which contains at least one N or S atom:

2. The method of claim 1, wherein each Ar is independently optionally substituted phenyl, 2-pyridyl, 3-pyridyl, 4-pyridyl, 2,6-pyridinyl or 3,5-pyrimidinyl.

3. The method of claim 1, wherein each Het is selected from the group consisting of

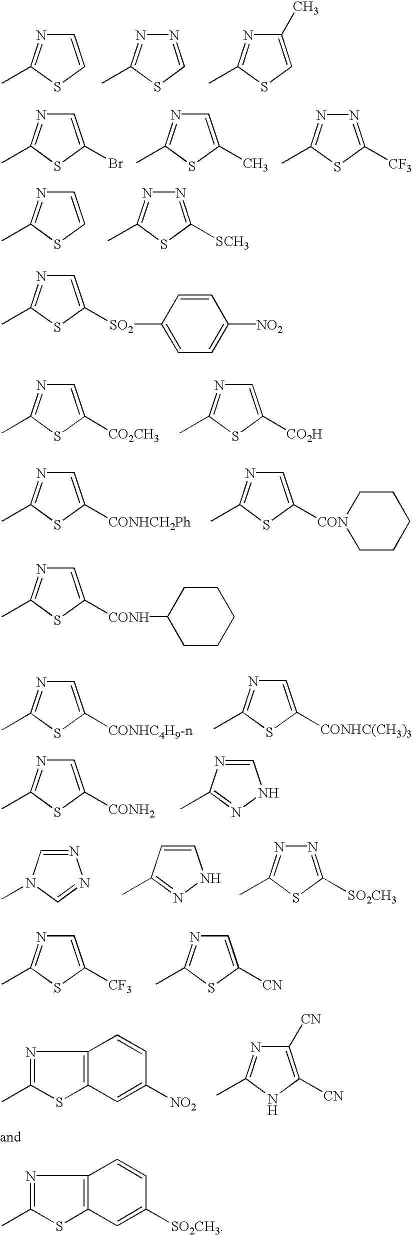

4. The method of claim 1, wherein the linker comprises an amide linkage.

5. The method of claim 1, wherein the substituents of said optionally substituted aryl are selected from the group consisting of halo, alkyl (1-6C) and alkoxy (1-6C)

6. The method of claim 1, wherein in formula (2), each aryl is linked through an oxygen to CH.

7. A pharmaceutical composition for use in treating conditions characterized by abnormal calcium channel activity which composition comprises, in admixture with a pharmaceutically acceptable excipient, a dosage amount of at least one compound of formula (1) or (2) or the salts thereof.
